(19) United States
(12) Patent Application Publication
(10) Pub. No.: US 2007/0264326 A1
(43) Pub. Date: Nov. 15, 2007

(54) Anti-misuse oral microparticle medicinal formulation

(75) Inventors: Florence Guimberteau, Montussan (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/648,605

(22) Filed: Jan. 3, 2007

Related U.S. Application Data
(63) Continuation of application No. 11/439,431, filed on May 24, 2006.

(30) Foreign Application Priority Data
May 24, 2005 (FR) 05 51344

(51) Int. Cl. A61K 9/58; A61K 9/16; A61K 9/48; A61K 9/62; A61K 9/64; A61K 9/60; A61K 9/50

(52) U.S. Cl. 424/454; 424/456; 424/458; 424/460; 424/461; 424/462; 424/489; 424/490; 424/492; 424/493; 424/494; 424/496; 424/498

(57) ABSTRACT

The field of the present invention is that of solid microparticulate analgesic oral medicines. The invention is that of providing novel analgesic medicines which allow at the same time the prevention of misuse and of addiction to certain analgesics, and/or the control of variability in the plasma concentration and/or the facilitation of oral administration; and/or the combination of analgesics with one another and/or with one or more active ingredients in the same oral form. The medicine according to the invention comprises (i) anti-misuse means and a plurality of microcapsules with modified release of analgesic(s), having a mean diameter of between 50 and 600 μm, (ii) at least 1000 microcapsules per dose; it being possible for this medicine to be administered once or twice a day for analgesic purposes.

### FIELD OF THE INVENTION

The field of the present invention is that of solid, microparticulate, analgesic oral medicinal formulations, the composition of which makes it possible, in particular, to reduce the number of daily doses taken, for analgesic purposes and to avoid misuse of the pharmaceutical active ingredient (AI) that they contain.

The AIs considered are more especially analgesics (AAIs). For the purpose of the present disclosure, the acronym “AAI” or “AI” denotes both a single active ingredient and a mixture of several active ingredients.

For the purpose of the present invention, the term “microparticulate medicinal formulation” is intended to mean any formulation in which the AAI is contained in microparticles less than 1000 microns in size. These particles containing the AAI may be microcapsules with modified release of AAI. In the latter case, the microcapsules are, for example, coated with at least one polymer coating which controls the rate of release of the AAI after oral administration.

### SITUATION OF THE PROBLEM

The use of analgesics poses several major public health problems.

The first problem (P1) is that a large number of analgesics are also narcotic products which induce addiction in patients. This addiction is in particular accentuated when the plasma concentration profile for the AAI exhibits very pronounced peaks and troughs. It would therefore be very advantageous to have a modified-release formulation which makes it possible to obtain a plasma concentration profile in the form of a “plateau” which levels out the peaks and troughs phenomena.

The second problem (P2) is related to the fact that certain immediate-release oral pharmaceutical formulations of AAI (IR formulations) produce erratic plasma profiles and do not guarantee an analgesic action which is homogeneous, effective and tolerable for all patients. In this way, some patients are incorrectly treated and/or, even more seriously, are victims of dangerous side effects. This great variability with premature and massive release of AAI can have serious consequences. Firstly, the patients for whom the concentration peak is early and of very large amplitude are victims of overdoses, which can be fatal. Secondly, the early decrease in plasma concentration after the peak is reflected by a very low AAI concentration level at the end of the period between two administrations. Thus, after having been subjected to an AAI overconcentration corresponding to the peak, the patients are insufficiently treated at the end of the period between two administrations. They are no longer under the effect of the AAI and therefore suffer from pain. Thirdly, this great variability leads the practitioner to limit the prescribed doses and certain patients can be incorrectly treated.

It would therefore be an advantage to have oral pharmaceutical formulations of AAI which make it possible to control the plasma concentration (in particular the maximum plasma concentration: Cmax) so as to avoid any massive and/or early and/or rapid release of the AAI.

The third problem (P3) is that of making it easier to administer an AAI-based medicine orally to populations who have trouble swallowing and are incapable of swallowing large tablets: infants, children, elderly individuals or patients suffering from highly incapacitating diseases such as cancers. It is clear that these difficulties in oral administration have a detrimental effect on treatment observance. Now, today, the only known suitable oral form consists of sachets of powder to be dispersed in a liquid. It would therefore be advantageous to have an oral form that is more convenient to use.

The fourth problem (P4) is that of the combination of several AAIs with one another, or even with other non-analgesic active ingredients AIs in the same pharmaceutical form. These combinations, which are sometimes useful in therapeutic terms, can sometimes be made difficult due to the chemical incompatibility (degradation) between two active ingredients and/or due to the need to have distinct release kinetics for the various AAIs and/or AIs in modified-release formulations.

The fifth problem (P5) is related to the fact that analgesics, in particular morphine derivatives, are often subjected to misuse. This misuse is intentional or unintentional abuse of AAI-based oral solid medicines, for any uses other than the therapeutic use(s) officially approved by the public health authorities concerned. Misuse is mainly encountered in the following cases:
a. addictive behavior (drug addiction, doping),
b. criminal behavior (chemical subjection),
c. use of a medicine in a manner not in accordance with the medical recommendations (posology), inadvertently or due to disabilities affecting the patient,
d. self-medication.

In case a. (or even in case b.), individuals who have the intention of misusing an oral solid medicine will generally apply themselves to making it either into a pulverulent form which can be inhaled or swallowed, or into a liquid form which can be injected using a syringe.

Obtaining an injectable liquid form from a solid oral medicine involves a step consisting of aqueous or organic extraction of the AAI targeted. This extraction is generally preceded by crushing.

Methods of administration by inhalation or by injection are particularly suitable for drug addicts because they are methods which make it possible to enhance the effects of the AAI and which promote its absorption in the body over short periods of time. When this powder is inhaled via the nose or dissolved in water and injected, the desired effects (doping effects or effects producing a feeling of euphoria) of the AAI manifest themselves very rapidly and in an exacerbated manner. Misuse of solid oral medicines can also be observed when the medicine is chewed before being swallowed, instead of being swallowed rapidly in accordance with the posology.

The risks associated with addictive behavior (a.) and criminal behavior (b.) and with self-medication (d.) are obvious. It will be recalled that the misuse of medicines by injection is a serious situation: the excipients can be responsible for local tissue necroses, for infections, and for respiratory and cardiac disorders.

As regards deviations (c.) of the use of a medicine related to inattention and/or to disabilities of the patient, they can also have serious consequences. For example, chewing AAI modified-release formulations before swallowing converts the medicine into an immediate-release form. Thus, at best the medicine is ineffective after a very short period of time, and at worst it becomes toxic.

This increasing phenomenon of misuse is becoming more and more worrying to health authorities, who are increasing appeals for the development of medicinal formulations for preventing abuse.

### PRIOR ART

Patent application EP-A-0647448 discloses an oral solid pharmaceutical formulation for the release of an opioid AAI (morphine) over a period of at least 24 h. The AAI is contained in microparticles of between 0.1 and 3 mm in size. These microparticles can each be formed by a matrix substrate containing the AAI and a hydrophobic compound. According to a variant, the microparticles are microcapsules of the reservoir type and are each formed by a nucleus comprising an inert core (sugar), coated with a layer containing AAI and excipients (lactose/polyvinypyrrolidone/hydroxypropylmethylcellulose (HPMC)) and with a layer for controlling the release of the AAI. This coating comprises, for example, a methacrylic copolymer (EUDRAGIT® RS 30D/tri-ethyl citrate/talc) or a cellulose-based polymer (ethylcellulose/methylcellulose/triethyl citrate/talc). An overcoating (AAI/HPMC) can be envisioned. These microcapsules gradually release the AAI over 24 h in an in vitro dissolution test, at 37° C. and at gastric pH.

U.S. Pat. No. 6,627,635 describes a pharmaceutical formulation with prolonged release over a period of 12 to 24 h, containing an opioid agonist (hydrocodone) and an antagonist (naltrexone) of the opioid, as anti-misuse means. This pharmaceutical formulation can be of matrix or reservoir type (AAI nucleus +coating controlling the diffusion of the AAI). It can be in the form of a tablet or of microparticles. The latter have a diameter of between 100 and 2500 μm (500-2000 μm). The coating is, for example, based on ethylcellulose and/or on a methacrylic copolymer (EUDRAGIT® RS30D and/or RL30D), and on an optional plasticizer (triethyl citrate). HPMC can be used in the coating or in an overcoating.

The oral solid pharmaceutical formulations according to EP-A-0647448 and U.S. Pat. No. 6,627,635 do not disclose pharmaceutical means for solving the abovementioned problems P1 to P5. In particular, EP-A-0647448 does not describe anti-misuse means—e.g. anti-crushing means—(problem P5). The anti-misuse means according to U.S. Pat. No.6,627,635—antagonist—are absolutely not satisfactory. This is because the antagonists of the AAI are substances which are pharmaceutically active and therefore potentially dangerous for users, and which can act against the normal use of the medicine.

### OBJECTIVES OF THE INVENTION

Under these circumstances, one of the essential objectives of the present invention is to make up for the deficiencies of the prior art.

Another essential objective of the invention is to provide novel oral, solid, analgesic medicines which allow, at the same time:
prevention of misuse, the latter being made very difficult or even impossible, in particular for the abovementioned cases (a.), (b.), (c.) and (d.), preferably without resorting to antagonists of the AAIs,
modified release of an AAI according to a plasma concentration profile in the form of a “plateau”, which levels out the peaks and troughs phenomena, and thus provides an advantageous solution to the major public health problem represented by addiction to certain AAIs;
and/or control of the variability of the plasma concentration (in particular the maximum plasma concentration: Cmax) so as to prevent a high inter- and/or intraindividual variability in the quality of the treatment;
and/or facilitation of administration for populations incapable of swallowing tablets which are often considerable in size, namely: infants, children and elderly individuals;
and/or combination of several AAIs with one another, or even with other non-analgesic active ingredients AIs in the same pharmaceutical formulation, even in the event of incompatibility between the substances in question and/or when the various AIs and/or AAIs must have distinct release kinetics;
and/or provision of an oral pharmaceutical formulations of AAI which can be administered one or more times daily and which offers the possibility of mixing the AAI with one or more active ingredients in the same oral form, with the possibility of readily and independently adjusting the release times of the various active ingredients.

An essential objective of the invention is to provide an oral pharmaceutical formulation of AAI which is used in such a way that it provides a quality of treatment which is more uniform and more reproducible from one patient to the other, compared with that which is proposed in the prior art.

Another essential objective of the present invention is to provide a means for reducing the inter- and/or intraindividual standard deviation of the maximum concentration Cmax of the plasma concentration profile.

Another essential objective of the invention is to provide an oral pharmaceutical formulation of AAI which reduces the inter- and/or intraindividual variability of the in vivo absorption of the AAI, which is a direct consequence of the sensitivity of certain modified-release oral pharmaceutical formulations (gastroretentive tablets, for example) with respect to the inter- and/or intraindividual variability of gastric emptying.

Another essential objective of the invention is to provide an oral pharmaceutical formulation of AAI which can be administered once or twice a day and is at least as effective as the immediate-release once-a-day formulations currently in use.

Another essential objective of the invention is to provide an oral pharmaceutical formulation of AAI which exhibits an in vitro dissolution profile independent of the dose of AAI.

Another essential objective of the invention is to provide an oral pharmaceutical formulation of AAI, whereby the microparticles of which it is composed have the same composition by weight irrespective of the therapeutic dose of AAI targeted.

Another essential objective of the invention is to provide an oral pharmaceutical formulation of AAI which can be administered once a day and which limits the risk of tissue deterioration due to local overconcentration of AAI.

Another essential objective of the invention is to provide an oral pharmaceutical formulation of AAI which can be administered once a day and which, despite the variability in solubility of the AAI in water as a function of the pH, releases the AAI according to the same kinetics, whether or not the patient is fasting.

Another essential object of the invention is to provide an oral pharmaceutical formulation of AAI which can exist in various pharmaceutical presentation formulations, including in particular: tablet, sachet, oral suspension, gelatin capsule, etc.

Another essential objective of the invention is to provide a novel oral solid medicine which makes it possible to avoid fraudulent abuse of the properties of the AI that it contains, by preventing any conversion of the medicine which would make it possible to take it orally, nasally and/or by injection (intravenous, subcutaneous, intramuscular, etc.) outside the therapeutic context. In so doing, the risks associated with these abuses would be prevented or, at the very least, greatly reduced.

Another essential objective of the invention is to provide a novel oral solid medicine which makes it possible to avoid misuse, while at the same time guaranteeing, for the patient normally followed up, a quality of treatment, in particular a dose, in accordance with said patient's needs.

Another essential objective of the invention is to provide a novel oral solid medicine which makes it possible to avoid misuse, without affecting the pharmacological properties of the medicine, and without causing the patient using the medicine normally, to run any additional risks, and finally, without being detrimental to the patient's comfort during administration.

Another essential objective of the invention is to provide a novel oral solid medicine which makes it possible to avoid misuse, which is simple to obtain, and for which the method of production does not cause its cost price to increase.

### BRIEF DESCRIPTION OF THE INVENTION

In order to attain these objectives, it is to the inventors' credit to have combined means for prolonged release of AAI capable of solving at least one of the problems P1 to P4, and specific anti-misuse means, i.e. anti-crushing means, capable of solving problem P5.

This combination was not self-evident. It was in fact necessary to develop means for controlling release of the AAI which are compatible with the anti-misuse means.

To this end, the inventors have had to reformulate the problem designated above as (P5) of the misuse of pharmaceutical formulations.

If the various methods of illicit administration of an active ingredient are examined, it appears in fact that crushing of the dry form is a required step.

In the case of misuse by nasal administration, the dry pharmaceutical formulation must, beforehand, be made into the form of a pulverulent powder which can be inhaled. Crushing of the pharmaceutical formulation is therefore clearly a required step.

In the case of misuse by oral administration of a prolonged-release dry form, it is necessary to accelerate the release of the active ingredient by finely crushing the microcapsules or the tablet.

In the case of misuse by parenteral administration, it is necessary, beforehand, to extract the AAI in a liquid phase, in practice water or organic solvents, at a concentration sufficiently high to avoid injecting volumes which are too large, for example greater than 1 ml. This extraction step is facilitated by a prior step of crushing the dry form in order to allow dissolution or suspension of the active ingredient. In addition, at the end of this extraction phase, misuse is only possible if the viscosity of the liquid is not too high (for example, less than or equal to 100 mPa.s).

Thus, crushing of a dry form is also a required step for the misuse of said pharmaceutical formulation by parenteral administration.

It is therefore to the Applicant's credit to have reformulated problem (P5) consisting of combating the misuse of dry pharmaceutical formulations, by distinguishing:
a subproblem (P5.1) consisting of preventing the crushing of the system containing the AI,
and a subproblem (P5.2) consisting of preventing the misuse of the AI after it has possibly been extracted.

This novel approach has allowed the Applicant to discover, surprisingly and unexpectedly, that it is advisable to involve, in the composition of the medicine the misuse of which it is sought to prevent, the AAI in the form of microcapsules and a combination of pharmaceutically acceptable excipients, which may or may not be in microparticulate form, and the method of physicochemical action that makes it possible to act against, or even to make impossible, any intentional or unintentional act of misuse.

Thus, the invention relates, in the main aspect, to an oral medicinal formulation comprising anti-misuse means and a plurality of microcapsules with modified release of at least one analgesic active ingredient (AAI), at least some of said microcapsules individually consisting of a nucleus comprising at least one AAI and coated with at least one coating for modified release of the AAI; the average diameter of said microcapsules being less than or equal to 1000 μm, preferably between 50 and 800 μm, more preferentially between 50 and 600 μm, and even more preferentially between 80 and 400 μm.

This multimicrocapsular medicinal formulation is novel and inventive in the following respect:
it comprises at least 1000 microcapsules per dose;
the amount of AAI and the modified-release coating are such that they allow administration in one or two doses a day for analgesic purposes;
and the anti-misuse means comprise anti-crushing means (a).

Preferably, the medicinal formulation according to the invention is free of antagonist agent(s) of the AAI.

The medicinal formulation according to the invention solves in particular the problem (P1) stated, i.e. that of the dependency of consumers with respect to AAIs, along with the incidental but no less important problem (P5) of misuse. It fulfils the objectives set effectively, simply and economically, by virtue of physicochemical means: use of coated microcapsules of AAI and of compatible anti-misuse means.

All these elements are completely harmless for the normal user. They are pharmacologically neutral (inert) compounds approved by the pharmacopoeia and by the public health authorities responsible for granting marketing authorizations for medicines.

Preferably, this medicinal formulation makes it possible to obtain, after taking one dose, a plasma profile defined as follows:
Cmax/C18h≦Cmax*/C18h*
preferably 1.5×Cmax/C18h≦Cmax*/C18h*
and even more preferentially
2.0×Cmax/C18h≦Cmax*/C18h*
with
C18h representing the plasma concentration of AAI, 18h after taking the dose,
C18h* representing the plasma concentration of AAI obtained under the same conditions as C18h, with a reference immediate-release oral pharmaceutical form, containing the same dose of AAI,
Cmax representing the maximum plasma concentration of AAI after taking the dose,
Cmax* representing the maximum plasma concentration of AAI obtained under the same conditions as Cmax, with a reference immediate-release oral pharmaceutical form, containing the same dose of AAI.

Preferably, this medicinal formulation is designed in such a way that it, and in particular the coating of the microcapsules, results in a decrease in the inter- and/or intraindividual standard deviation of the Cmax when it is administered orally to a sample of individuals, whatever the fed state or fasting state of the individuals, compared with a pharmaceutical formulation with immediate release of AAI administered to this same sample of individuals, at the same dose, which makes it possible to ensure a smaller variability in effectiveness and in therapeutic safety of the pharmaceutical form.

One of the characteristics of the medicinal formulation according to the invention is thus defined through a reference clinical test in which the formulation is administered orally to a sample of human individuals, under experimental conditions which may, for example, be as follows: administration of the formulation (gelatin capsule, tablet or suspension) once a day, at a given dose, after breakfast to 20 normal volunteers in the course of a crossover trial study. The plasma concentrations of AAI are measured at times: 0-0.25-0.5-0.75-1-1.5-2-3-4-6-8-10-12-16-18-20-24-36-48 hours post-administration.

This clinical test defines the invention in terms of the pharmacokinetic properties obtained specifically under the conditions of the test. Nevertheless, the invention is not limited to an implementation under the conditions of this reference clinical test.

The factor (f) of decrease in the inter- and/or intraindividual standard deviation of the Cmax is defined as being the ratio of the inter- and/or intraindividual standard deviation of the Cmax of the reference immediate-release pharmaceutical formulation, to the inter- and/or intraindividual standard deviation of the Cmax of the pharmaceutical formulation according to the invention, administered at the same dose of AAI.

Advantageously, the factor (f) of decrease in the inter- and/or intraindividual standard deviation of the Cmax is defined as follows: f≧1.05; preferably, f≧1.5; and, even more preferentially, f is between 2.0 and 20.

For the purpose of the present invention, the mean peak/trough modulation—PTM—of the plasma profile of an AI is defined in the following way: on each of the individual plasma profiles, the individual maximum concentration Cmax′ and the concentration cT′ are measured, T hours after a single oral administration. The PTM is the arithmetic mean of the Cmax′/cT′ individual ratios.

For a product intended to be administered daily to the patient, T is 24 hours after the single administration. If the concentration cT′ (T=24 h) is below the limit of detection of the assay method used and below the limit of detection of the method recommended by the Pharmacopeia of the United States of America and/or known to those skilled in the art, the concentration c24′ used to calculate the PTM will be replaced with the concentration cx′ measured x hours after oral administration, x being the most belated hour at which it is possible to measure a concentration above the limit of detection of the method used. In this case, x is less than 24 hours after single administration. For example, x is equal to 18 h, or, failing this, 12 h.

For a product intended to be administered twice daily to the patient, T is 12 hours in single administration. Here also, if the concentration cT′ (T=12 h) is below the limit of detection of the assay method used and below the limit of detection of the method recommended by the Pharmacopeia of the United States of America and/or known to those skilled in the art, the concentration c12′ used to calculate the PTM will be replaced with the concentration cx′ measured x hours after oral administration, x being the most belated hour at which it is possible to measure a concentration above the limit of detection of the method used. In this case, x is less than 12 hours after single administration.

The medicinal formulation according to the invention is designed in such a way that, when it is administered orally to a sample of individuals, it results in a mean peak/trough modulation of the plasma profiles of the AAI less than or equal to the mean peak/trough modulation of the AAI of the same sample of individuals having received the same dose of an immediate-release AAI form.

For the purpose of the invention, the reduction in the peak/trough modulation of the plasma concentration profiles is given, for example, by the peak/trough modulation decrease factor g. The factor g is defined by the ratio of the peak/trough modulation of the reference immediate-release formulation to the peak/trough modulation of the formulation involved in the use according to the invention.

Preferably, the peak/trough modulation decrease factor g is such that: g≧1.05; preferably, g≧1.5; and, even more preferentially, g is between 2.5 and 20.

In accordance with the use according to the invention, the coating or the matrix of the pharmaceutical formulation is designed in such a way that the oral administration of this formulation, to a sample of individuals, results in a variability of the peak/trough modulation of the plasma profiles of the AAI which is less than the variability of the peak/trough modulation of the AI of the same sample of individuals having received the same dose of an immediate-release AI form.

For the purpose of the invention, the reduction in the variability of the peak/trough modulation of the plasma concentration profiles is given, for example, by the factor g′ for decrease in the standard deviation of the peak/trough modulation. The factor g′ is defined by the ratio of the standard deviation of the peak/trough modulation of the reference immediate-release formulation to the standard deviation of the peak/trough modulation of the formulation involved in the use according to the invention.

Preferably, the factor g′ for decrease in the standard deviation of the peak/trough modulation is such that: g′≧1.1; preferably, g′≧1.5; and, even more preferentially, g′ is between 2.5 and 20.

This medicinal formulation with modified release of AAI is also designed in such a way that the microcapsules, once ingested, are dispersed and individualized when they reach the stomach, which guarantees regular and gradual gastric emptying of the microunits, in the fed state just as in the fasting state, and therefore, ultimately, release of the AAI within its gastrointestinal window of bioabsorption.

Definitions for the purpose of the invention:
The term “dose” denotes, for the purpose of the invention, the amount of AAI contained in the medicinal formulation administered orally;
The term “immediate release” denotes, in the present disclosure, the release, by an immediate-release formulation (IRF), of most of the amount of AAI in a relatively brief period of time, for example:
   at least 70% of the AAI is released in vivo in one hour, preferably in thirty minutes, after oral ingestion;
   or at least 70% of the AAI is released in one hour, preferably in thirty minutes, at any pH of between 1.4 and 6.8 in an in vitro dissolution test.

All the dissolution profiles to which reference is made in the present disclosure are realized according to the indications of the European Pharmacopoeia, 4th edition, entitled: “Dissolution test for solid oral formulations”: type II dissolutest carried out under SINK conditions at 37° C. and with stirring at 100 rpm.
The term “modified release” denotes, in the present disclosure, the release of AAI by an oral pharmaceutical formulation, occurring in vivo at a rate less than that of a reference “immediate-release formulation”, IRF*. Such a modified-release formulation can, for example, comprise an immediate-release phase and a slow-release phase. Modified-release formulations are well known in this field; see, for example, Remington: The science and practice of pharmacy, 19th edition, Mack Publishing Co., Pennsylvania, USA. The modified release can in particular be a prolonged and/or controlled, or even delayed, release.
The pharmacokinetic parameters to which reference is made in the present invention are defined in the following way. After oral administration of the pharmaceutical formulation to a sample of N human individuals, the individual plasma concentration profile is measured in each of the patients, from which the individual pharmacokinetic parameters are drawn: Tmax, Cmax, C18h:
   Tmax is the amount of time after which the plasma concentration reaches its maximum, Cmax.
   C18h is the plasma concentration 18 hours after administration.

Based on these individual parameters, those skilled in the art conventionally calculate the mean values of these parameters and their standard deviations. Further details on the discussion of these parameters will be found in the work: Pharmacokinetics and Pharmacodynamic Data Analysis, 3rd ed., J. Gabrelsson et al., Kristianstads Bocktryckeri AB, Sweden, 2000.
The comparison of the parameters C18h and C18h*, and Cmax and Cmax* is carried out in a statistically significant manner, under the same conditions and at the same dose of AAI.
The peak/trough modulation of the plasma concentration profiles is defined by the mean of the Cmax/C18h ratio for the AAI.
The expression “dispersed and individualized” means that the AAI-based microcapsules are not trapped in a matrix when they reach the stomach just after they have been ingested. The microcapsules become disseminated in the stomach after they have entered the latter.

Advantageously, the medicinal formulation according to the invention comprises microgranules with immediate release of AAI.

The secondary advantages of the invention are in particular as follows:
This oral pharmaceutical formulation of AAI, which can be administered once or twice a day, is such that, once ingested, the AAI that it contains is released in the gastrointestinal tract and bioabsorbed within its absorption window, even if the latter is narrow.
This oral pharmaceutical formulation of AAI, which can be administered once or twice a day, guarantees that, once the oral pharmaceutical formulation has been ingested, the AAI that it contains will not pass in front of its bioabsorption window without being released.
This oral pharmaceutical formulation of AAI, which can be administered once or twice a day, guarantees that, once the oral pharmaceutical formulation has been ingested, the AAI that it contains will be released independently of the open or closed state of the pylorus.
This oral pharmaceutical formulation of AAI, which can be administered once or twice a day, is not subject, or barely subject, to the phenomenon of inter- and/or intraindividual variability of gastric emptying and, ultimately, of in vivo absorption of AAI.
This oral pharmaceutical formulation of AAI, which can be administered once or twice a day, is at least as effective as the immediate-release once-a-day formulations currently in use.
This oral pharmaceutical formulation of AAI, which can be administered once or twice a day and which comprises microcapsules with modified release of AAI, draws some of its advantages from the small size (≦1000 μm) of these microcapsules and the large number thereof (e.g. at least a thousand or so per dose), which allows gradual and well-controlled gastric emptying.
This oral pharmaceutical formulation of AAI, which can be administered once or twice a day, makes it possible to increase the Tmax of the AAIs and also the period of time for which the plasma concentration of AAI is greater than the minimum plasma concentration of AAI, below which the AAI is therapeutically ineffective.
This oral pharmaceutical formulation of AAI exhibits an in vitro dissolution profile independent of the dose of AAI.
This oral pharmaceutical formulation of AAI is composed of microparticles which have the same composition by weight irrespective of the doses of AAI.
This oral pharmaceutical formulation of AAI, which can be administered once or twice a day, is suitable for patients who have difficulty in swallowing, in particular for children or infants who not only cannot swallow, but who, in addition, require an adjustment of the dose administered according to their weight.
This oral pharmaceutical formulation of AAI, which can be administered once or twice a day, offers the possibility of mixing the AAI with one or more other active ingredients in the same oral form, it being possible for the respective release times of these various active ingredients to be readily adjusted, independently of one another.
This oral pharmaceutical formulation of AAI can exist in various pharmaceutical presentation formulations, including in particular: tablet, sachet, oral suspension, gelatin capsule, etc.
The oral pharmaceutical formulation according to the invention consists-of a large number (for example, of the order of about one to several thousand) of microcapsules (or microgranules with immediate release of AAI, or of a mixture of several types of microcapsules or microgranules), this multiplicity ensuring, statistically, good reproducibility of the kinetics of transit of the AAI throughout the gastrointestinal tract, and, subsequently, good control of bioavailability and better effectiveness.
The use of a mixture of microcapsules with different modified release profiles makes it possible to produce release profiles which ensure, by means of suitable regulation of the various fractions, a constant level of plasma concentration of AAI.
There is less sensitivity to the variability in gastric emptying because the emptying, which takes place over a large number of particles, is statistically more reproducible.
Bringing tissues into contact with a high dose of AAI (dose dumping) is prevented. Each microcapsule in fact contains only a very small dose of AAI. The risk of tissue deterioration due to a local overconcentration of AAI is thus done away with.
This pharmaceutical formulation does not induce any degradation of the starting AAI and preserves the initial polymorphism of the AAI.
Their size of less than or equal to 1000 μm and also the characteristics of their possible coating allows the microcapsules to increase their transit time in the upper parts of the gastrointestinal tract, which ensures an increase in the amount of time taken for the AAI to pass in front of its absorption window and thus maximizes the bioavailability of the AAI.

In accordance with a first embodiment of the invention, in the medicinal formulation 70% of the AAI is released between 1 and 24 h, preferably 2 and 15 h, and more preferentially 2 and 12 h.

Advantageously, this medicinal formulation has an in vitro dissolution profile of the oral pharmaceutical formulation such that, for any value of the time t of between 2 h and t(70%), preferably for any value of the time t of between 1 h and t(70%), the percentage of AAI dissolved is greater than or equal to 35 t/t(70%).

The composition of the coating of the microcapsules according to the first embodiment corresponds, advantageously, to one of the following two families A and B:

Family A:

1A—at least one film-forming polymer (Pol. 1) which is insoluble in the fluids of the tract, present in a proportion of 50% to 90%, preferably 50% to 80% by weight on a dry basis relative to the total mass of the coating composition, and comprising at least one water-insoluble derivative of cellulose;

2A—at least one nitrogenous polymer (Pol. 2) present in a proportion of 2% to 25%, preferably 5% to 15% by weight on a dry basis relative to the total mass of the coating composition and consisting of at least one polyacrylamide and/or one poly-N-vinylamide and/or one poly-N-vinyllactam;

3A—at least one plasticizer present in a proportion of 2% to 20%, preferably of 4% to 15% by weight on a dry basis relative to the total mass of the coating composition and consisting of at least one of the following compounds: glyceryl esters, phthalates, citrates, sebacates, cetyl alcohol esters, castor oil;

4A—at least one surfactant and/or lubricant, present in a proportion of 2% to 20%, preferably of 4% to 15% by weight on a dry basis relative to the total mass of the coating composition and chosen from anionic surfactants and/or from nonionic surfactants and/or from lubricants; it being possible for said surfactant and/or lubricant to comprise just one or a mixture of the abovementioned products;

Family B:
1B—at least one film-forming polymer which is insoluble in the fluids of the gastrointestinal tract,
2B—at least one water-soluble polymer,
3B—at least one plasticizer,
4B—and, optionally, at least one surfactant/lubricant, preferably consisting of at least one anionic surfactant and/or at least one nonionic surfactant.

According to a preferred mode of the invention, the families A and B from which the constituents of the coating composition are chosen are as follows:

Family A

1A—ethylcellulose and/or cellulose acetate;

2A—polyacrylamide and/or polyvinylpyrrolidone;

3A—castor oil;

4A—alkali metal or alkaline earth metal salt of fatty acids, stearic acid and/or oleic acid being preferred, a polyoxyethylenated sorbitan ester, derivatives of polyoxyethylenated castor oil, a stearate, preferably calcium stearate, magnesium stearate, aluminum stearate or zinc stearate, a stearyl fumarate, preferably sodium stearyl fumarate, glycerol behenate; taken on their own or as a mixture with one another;

Family B:

1B

water-insoluble derivatives of cellulose, ethylcellulose and/or cellulose acetate being particularly preferred, water-insoluble acrylic polymers, polyvinyl acetates; and mixtures thereof;

2B

water-soluble derivatives of cellulose, polyacrylamides, poly-N-vinylamides, poly-N-vinyllactams, polyvinyl alcohols (PVAs), polyoxyethylenes (POEs), polyvinylpyrrolidones (PVPs) (the latter being preferred), and mixtures thereof;

3B

glycerol and its esters, preferably from the following subgroup: acetylated glycerides, glyceryl monostearate, glyceryl triacetate, glyceryl tributyrate, phthalates, preferably from the following subgroup: dibutyl phthalate, diethyl phthalate, dimethyl phthalate, dioctyl phthalate,citrates, preferably from the following subgroup: acetyl tributyl citrate, acetyl triethyl citrate, tributyl citrate, triethyl citrate, sebacates, preferably from the following subgroup: diethyl sebacate, dibutyl sebacate, adipates, azelates, benzoates, plant oils, fumarates, preferably diethyl fumarate, malates, preferably diethyl malate, oxalates, preferably diethyl oxalate, succinates, preferably dibutyl succinate, butyrates, cetyl alcohol esters, salicylic acid, triacetin, malonates, preferably diethyl malonate, castor oil (the latter being particularly preferred), and mixtures thereof;

4B

alkali metal or alkaline earth metal salts of fatty acids, stearic acid and/or oleic acid being preferred, polyoxyethylenated oils, preferably polyoxyethylenated hydrogenated castor oil, polyoxyethylene-polyoxypropylene copolymers, polyoxyethylenated sorbitan esters, polyoxyethylenated castor oil derivatives, stearates, preferably calcium stearate, magnesium stearate, aluminum stearate or zinc stearate, stearyl fumarates, preferably sodium stearyl fumarate, glycerol behenate, and mixtures thereof.

Preferably, the film coating consists of a single layer, the mass of which represents from 1% to 50% by weight, preferably from 5% to 40% by weight, of the total mass of the microcapsules.

Other details and examples of compositions and of methods for obtaining the microcapsules according to the first embodiment of the invention are given in WO-A-03/084518, the contents of which are incorporated by reference into the present disclosure.

For further details in qualitative and quantitative terms, as regards the coating composition of family A, reference is made to European patent EP-B-0 709 087, the content of which are incorporated by reference into the present disclosure.

In accordance with a second embodiment of the invention, the oral medicinal formulation is such that:

the release of AAI is controlled by two distinct triggering mechanisms, one being based on a variation in pH and the other allowing the release of AAI after a predetermined residence time in the stomach;
at constant pH 1.4, the dissolution profile contains a lag phase which lasts 7 hours or less, preferably 5 hours or less, and even more preferentially between 1 and 5 hours,
and the passing from pH 1.4 to pH 7.0 results in a release phase which begins without any lag time.

In accordance with the second embodiment of the invention, the pharmaceutical formulation has an in vitro dissolution profile which may be as indicated below:
at least 20% of the AAI is released after 2 hours at pH=1.4;
at least 50% of the AAI is released after 16 hours at pH=1.4.

Advantageously, the microcapsules with modified release of AAI, according to the second embodiment of the invention, have the following specificities:
the coating allowing the modified release of AAI comprises a composite material containing:
   at least one hydrophilic polymer I bearing groups which are ionized at neutral pH,
   at least one hydrophobic compound II; representing a mass fraction (% by weight relative to the total mass of the microcapsules)≦40; and
their mean diameter is less than 2000 μm, and preferably between 50 and 800 μm, and even more preferentially between 100 and 600 μm.

According to another advantageous characteristic, the composite material I-II of the coating allowing the modified release of AAI is such that:
the ratio by weight II/I is between 0.2 and 1.5, preferably between 0.5 and 1.0,
and the hydrophobic compound II is selected from products which are crystalline in the solid state and which have a melting point T_{mII}≧40° C., preferably T_{mII}≧50° C., and even more preferentially 40° C.≦T_{mII}≦90° C.

According to one embodiment of predilection, the hydrophilic polymer I is chosen from:
I.a copolymers of (meth)acrylic acid and of an alkyl ester of (meth)acrylic acid, and mixtures thereof;
I.b cellulose derivatives, preferably cellulose acetates, cellulose phthalates, cellulose succinates, and mixtures thereof, and even more preferentially hydroxypropylmethylcellulose phthalates, hydroxypropylmethylcellulose acetates, hydroxypropylmethylcellulose succinates, and mixtures thereof;
and mixtures thereof.

The polymers I which are even more preferred are copolymers of (meth)acrylic acid and of alkyl (e.g. C₁-C₆ alkyl) esters of (meth)acrylic acid. These copolymers are, for example, of the type such as those sold by the company Röhm Pharma Polymers under the registered trade marks EUDRAGIT®, of the series L and S (such as, for example, EUDRAGIT® L100, S100, L30 D-55 and L100-55). These copolymers are anionic enteric copolymers which are soluble in an aqueous medium at pHs greater than those encountered in the stomach.

Still according to the embodiment of predilection, the compound II is chosen from the group of following products:

II.a plant waxes taken on their own or as mixtures with one another;

II.b hydrogenated plant oils taken on their own or as mixtures with one another;

II.c mono- and/or di- and/or triesters of glycerol and of at least one fatty acid;

II.d mixtures of monoesters, of diesters and of triesters of glycerol and of at least one fatty acid;

II.e and mixtures thereof.

Even more preferably, the compound II is chosen from the group of following products: hydrogenated cottonseed oil, hydrogenated soybean oil, hydrogenated palm oil, glycerol behenate, hydrogenated castor oil, tristearin, tripalmitin, trimyristin, yellow wax, hard fat or fat useful as suppository bases, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, omega-3, and any mixture with one another, preferably, from the subgroup of following products: hydrogenated cottonseed oil, hydrogenated soybean oil, hydrogenated palm oil, glycerol behenate, hydrogenated castor oil, tristearin, tripalmitin, trimyristin, and any mixture with one another.

In practice, and without this being limiting, the compound II is preferably chosen:
from the group of products sold under the following tradenames: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostéarique®, Geleol®, Hydrine®, Monthyle®, and mixture thereof;
and also from the group of additives for which the codes are as follows: E 901, E 907, E 903, and mixtures thereof;
and, preferably from the group of products sold under the following trade names: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab®, and mixtures thereof.

According to another advantageous characteristic of the invention, the coating which allows the modified release of AAI is free of talc.

Advantageously, the coating of the microcapsules can comprise, in addition to the essential constituents I and II, other conventional ingredients known to those skilled in the art, such as, in particular:
colorants,
plasticizers, such as, for example, dibutyl sebacate,
hydrophilic compounds, such as, for example, cellulose and its derivatives and polyvinylpyrrolidone and its derivatives,
and mixtures thereof.

Without it being limiting, and according to an embodiment which is even more preferred, the coating of the microcapsules with modified release of AAI comprise a single composite I-II film coating.

Other details and examples of compositions and of methods for obtaining the microcapsules according to the second embodiment according to the invention are given in WO-A-03/030878, the contents of which are incorporated by reference into the present disclosure.

In quantitative terms, the monolayer of coating can represent, for example, at most 40%, preferably at most 30% by weight of the microcapsules. Such a limited degree of coating makes it possible to produce pharmaceutical units which each contain a high dose of active ingredient, without exceeding a completely unacceptable size with regard to swallowing. The observance and therefore the success of the treatment can only be improved thereby.

According to a third embodiment of the invention, the oral pharmaceutical formulation according to the invention comprises at least two populations of microcapsules with modified release of AAI. Each population of microcapsules with modified release of AAI can be in accordance with the first or with the second embodiment of the invention.

According to a variant -2i- of the second embodiment of the invention combined with the third embodiment, the oral pharmaceutical formulation according to the invention comprises at least two populations of microcapsules having different dissolution profiles, for at least one pH value of between 1.4 and 7.4.

According to a variant -2ii- of the second embodiment of the invention combined with the third embodiment, the oral pharmaceutical formulation according to the invention comprises at least two populations of microcapsules with modified release of AAI which differ by virtue of their respective triggering pHs.

According to another variant -2iii- of the second embodiment of the invention combined with the third embodiment, the oral pharmaceutical formulation according to the invention comprises at least two populations of microcapsules with modified release of AAI which differ by virtue of their respective triggering times.

According to a fourth embodiment of the invention, the oral pharmaceutical formulation according to the invention comprises at least one population of microcapsules with modified release of AAI and at least one population of microgranules with immediate release of AAI.

According to a variant -2iv- of the second embodiment of the invention combined with the fourth embodiment, the oral pharmaceutical formulation according to the invention comprises:
at least one population of microgranules with immediate release of AAI;
at least one population Pol. 1 of microcapsules with modified release of AAI, and
at least one population Pol. 2 of microcapsules with modified release of AAI;
   and, moreover, the respective triggering pHs of P1 and of P2 differ by at least 0.5 of a pH unit, preferably by at least 0.8 of a pH unit, and even more preferentially by at least 0.9 of a pH unit.

Advantageously, the respective triggering pHs of the various populations of microcapsules with modified release of AAI are between 5 and 7.

According to a variant -2v- of the second embodiment of the invention combined with the fourth embodiment, the oral pharmaceutical formulation according to the invention comprises:
at least one population of microgranules with immediate release of AAI;
at least one population Pol. 1′ of microcapsules with modified release of AAI, the triggering pH of which is equal to 5.5; and
at least one population Pol. 2′ of microcapsules with modified release of AAI, the triggering pH of which is between 6.0 inclusive and 6.5 inclusive.

The populations Pol. 1, Pol. 2, Pol. 1′ and Pol. 2′ of the variants -2iv- and -2v- of the second embodiment comprise microcapsules with modified release of AAI, obtained in accordance with the second embodiment of the invention.

To illustrate the variants according to which microgranules with immediate release of AAI are present in the pharmaceutical formulation according to the invention, it can be specified that these variants can correspond to cases where this pharmaceutical formulation comprises, for example, at least one population of microgranules with immediate release of AAI, the behavior of which in an in vitro dissolution test is such that at least 80% of AAI is released in 1 hour at any pH between 1.4 and 7.4.

Advantageously, the AAI is selected from opiates, and more particularly from the group comprising the following compounds: anileridine, acetorphine, acetyl-alpha-methylfentanyl, acetyldihydrocodeine, acetylmethadol, alfentanil, allylprodine, alphacetylmethadol, alphameprodine, alphaprodine, alphamethadol, alphamethylfentanyl, alpha-methylthiofentanyl, alphaprodine, anileridine, atropine, butorphanol, benzethidine, benzylmorphine, beta-hydroxyfentanyl, beta-hydroxymethyl-3-fentanyl, betacetylmethadol, betameprodine, betamethadol, betaprodine, bezitramide, buprenorphine, dioxaphetyl butyrate, clonitazene, cyclazocine, cannabis, cetobemidone, clonitazene, codeine, coca, cocaine, codoxime, poppy straw concentrate, dezocine, dimenoxadol, dioxaphetyl butyrate, dipipanone, desomorphine, dextromoramide, dextropropoxyphene, diampromide, diethylthiambutene, difenoxine, dihydrocodeine, dihydroetorphine, dihydromorphine, dimenoxadol, dimepheptanol, dimethylthiambutene, diphenoxylate, dipipanone, drotebanol, eptazocine, ethoheptazine, ethylmethylthiambutene, ethylmorphine, etonitazene, ecgonin, ephedrine, ethylmethyl-thiambutene, ethylmorphine, etonitazene, etorphine, etoxeridine, fentanyl, furethidine, heroin, hydrocodone, hydromorphinol, hydromorphone, hydroxypethidine, isomethadone, ketobemidone, levallorphan, lofentanil, levomethorphan, levomoramide, levophenacylmorphan, levorphanol, meptazinol, meperidine, metazocine, methadone, methyldesorphine, methyldihydromorphine, methylphenidate, methyl-3-thiofentanyl, methyl-3-fentanyl, metopon, moramide, morpheridine, morphine, MPPP, myrophine, nalbuphine, narceine, nicomorphine, norlevorphanol, normethadone, nalorphine, normorphine, nicocodine, nicodicodine, nicomorphine, noracymethadol, norcodeine, norlevorphanol, normethadone, normorphine, norpipanone, opium, oxycodone, oxymorphone, papaveretum, phenadoxone, phenoperidine, promedol, properidine, propiram, propoxyphene para-fluorofentanyl, PEPAP, pentazocine, pethidine, phenampromide, phenazocine, phenomorphan, phenoperidine, pholcodine, piminodine, piritramide, proheptazine, propanolol, properidine, propiram, racemethorphan, racemoramide, racemorphan, remifentanil, sufentanil, thebacone, thebaine, thiofentanyl, tilidine, trimeperidine, tramadol, pharmaceutically acceptable salts of these compounds and mixtures of these compounds and/or of their salts.

The oral medicinal formulations according to the invention can comprise at least one other active ingredient other than an AAI. The abbreviation AI will denote hereinafter, without distinction, one or more active ingredients other than an AAI.

The in vivo or in vitro release of the AI can be immediate or modified. The AI can be contained in microgranules with immediate release of the AI or in microcapsules with modified release of the AI.

This AI can be chosen, inter alia, from the group comprising antidepressants, amphetamines, anorexigens, antalgics, antiepileptics, antimigraine agents, antiparkinsonian agents, antitussives, anxiolytics, barbiturics, benzodiazepines, hypnotics, laxatives, neuroleptics, psychostimulants, psychotropic agents, sedatives, stimulants, anti-inflammatories, pharmaceutically acceptable salts of these compounds and mixtures of these compounds and/or of their salts.

By way of examples of anti-inflammatories, mention may be made of ibuprofen, acetaminophen, diclofenac, naproxene, benoxaprofen, flurbiprofen, fenoprofen, flubufen, ketoprofen, indoprofen, piroprofen, carprofen, oxaprozine, pramoprofen, muroprofen, trioxaprofen, suprofen, aminoprofen, tiaprofenic acid, fluprofen, bucloxic acid, indomethacin, sulindac, tolmetine, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, pharmaceutically acceptable salts of these compounds and mixtures of these compounds and/or of their salts.

In accordance with an advantageous mode of the invention, the medicinal formulation comprises at least two populations of microcapsules having different release profiles according to the similarity factor f2.

Advantageously, the anti-misuse means of the medicinal formulation comprise means (b) envisioned for preventing misuse of the AAI after a possible liquid extraction.

Preferably, the medicinal formulation of the invention is free of antagonist agent(s) of the AAI.

Preferably, the anti-crushing means (a) of the medicinal formulation comprise:
an overcoating for protecting the microcapsules of AAI, having at least one of the following characteristics:
   viscoelastic properties so as to absorb the energy dissipated during crushing,
   a low cohesive strength so as to promote breaking of the overcoating and not of the microcapsules,
   a low surface energy so as to promote sliding of the microcapsules during crushing,
   an ability to form a paste under strong shear,
and/or excipients in the free state, i.e. not contained in or supported by microcapsules and capable of acting against, or even preventing, the crushing of the microcapsules of AAI.

Advantageously, the overcoating for protecting the microcapsules for release of AAI is designed in such a way that it makes it possible, in the event of crushing, to maintain a non-immediate release for at least some of said microcapsules with modified release of AAI.

More specifically, the overcoating for protecting the microcapsules of AAI preferably comprises:
at least one film-forming compound which ensures the cohesion of the overcoating and at least one of the following three compounds:
a lubricant/caking agent,
a viscoelastic compound,
a plasticizer.

In practice, the film-forming compound (i) is, for example, chosen from:
cellulose derivatives
acrylic polymers
and mixtures thereof.

As regards the lubricant/caking agent (ii), it is more especially chosen from the group comprising:
stearic acid and stearates, preferably calcium stearate, zinc stearate or magnesium stearate;
magnesium oxide;
poloxamers;
sodium benzoate;
anionic, cationic or nonionic surfactants;
starches, preferably corn starch;
talc;
colloidal silica;
waxes, preferably hydrogenated plant oils; and even more preferentially hydrogenated cottonseed oils, hydrogenated soybean oils, hydrogenated palm oils, glycerol behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas-3, and mixtures of all or some of these waxes; and/or
fatty bases for suppositories, comprising glycerine, triglycerides, theobroma oils, cocoa butters, and mixtures of all or some of these products;
and mixtures thereof.

The viscoelastic agent (iii) is for its part, preferably selected from the group of following products:
poly-N-vinylamides,
gum bases,
fatty alcohols,
poly-N-vinyllactams,
polyvinyl alcohols (PVAs),
polyoxyethylenes (POEs),
polyethylene glycols (PEGs),
polydextroses,
hydrogenated mono-, di- and polysaccharides,
polyvinylpyrrolidones (PVPs) (the latter being preferred),
and mixtures thereof.

Preferably, the plasticizer (iv) is selected from the group of following products:
glycerol and its esters, preferably from the following subgroup: acetylated glycerides, glyceryl monostearate, glyceryl triacetate, glyceryl tributyrate,
phthalates, preferably from the following subgroup: dibutyl phthalate, diethyl phthalate, dimethyl phthalate, dioctyl phthalate,
citrates, preferably from the following subgroup: acetyl tributyl citrate, acetyl triethyl citrate, tributyl citrate, triethyl citrate,
sebacates, preferably from the following subgroup: diethyl sebacate, dibutyl sebacate,
adipates,
azelates,
benzoates,
plant oils, preferably cottonseed oils, soybean oils, palm oils, castor oils, and mixtures of all or some of these oils;
fumarates, preferably diethyl fumarate,
malates, preferably diethyl malate,
oxalates, preferably diethyl oxalate,
succinates, preferably dibutyl succinate,
butyrates,
cetyl alcohol esters,
triacetin,
malonates, preferably diethyl malonate,
and mixtures thereof.

The excipients optionally included in the anti-crushing means (a) are chosen rather from:
compression agents,
and/or inert microbeads,
and/or gum bases,
and/or viscoelastic agents of the type such as those defined above.

Preferably, the means (b) envisioned for preventing misuse of the AI after a possible liquid extraction comprise “viscosifier” excipients capable of increasing the viscosity of the extraction liquid so as to act against misuse, in particular by injection.

These “viscosifier” excipients capable of increasing the viscosity of the extraction liquid so as to act against misuse, in particular by injection, are preferably present:
in and/or on microcapsules,
and/or in an overcoating of all or some of the microcapsules of AAI,
and/or in the free state, i.e. not contained in or supported by microcapsules.

It is more particularly advantageous to envision, in the medicinal formulation according to the invention, “viscosifier” excipients capable of increasing the viscosity of the liquid used for the possible extraction, according to kinetics similar to the kinetics of extraction of the AAI contained in the microcapsules, so as to trap the AAI extracted, in the viscious medium.

In practice, the excipients included in the means (b) are, for example, chosen from the groups of following polymers:
polyacrylic acids and their derivatives, and/or
polyoxyethylenes (POEs), and/or
polyvinyl alcohols (PVAs),
polyvinylpyrrolidones (PVPs), and/or
gelatins, and/or
cellulose derivatives (e.g. hydroxypropylmethylcellulose, methylcellulose, hydroxyethylcellulose, carboxymethylcellulose, hydroxypropylcellulose), and/or
polysaccharides, preferably from the subgroup comprising: sodium alginate, pectins, guars, xanthans, carrageenans, gellans,
and mixtures thereof.

The medicinal formulation according to the invention has at least one of the following notable characteristics:
it cannot be converted (or is difficult to convert) into a dry form which can be administered by nasal aspiration;
it cannot be converted (or is difficult to convert) into an injectable form;
extraction of the AAI by chewing and/or crushing is not effective (or only with difficulty).

The medicinal formulation according to the invention can comprise, in addition to the microunits consisting of microcapsules with modified release of AAI, microunits of AAI other than microcapsules, i.e. microgranules with immediate release of AAI and/or of one or more other active ingredient(s) AI. These immediate-release microgranules are advantageously uncoated and may be of the same type as those that are useful in the preparation of the microcapsules according to the invention.

In addition, the assembly of microunits (microcapsules and optionally microgranules) constituting the medicine according to the invention can be formed by various populations of microunits, these populations differing from one another at least by virtue of the nature of the active ingredient(s) other than the AAI contained in these microunits and/or by virtue of the amount of AAI or of other optional active ingredient(s) that they contain and/or by virtue of the composition of the coating and/or by virtue of the fact that they are modified-release or immediate-release.

According to a specific embodiment, the medicinal formulation according to the invention is in the form of a single daily oral dose comprising from 1000 to 500 000 microunits containing AAI.

According to another specific embodiment, the medicinal formulation according to the invention is in the form of a single daily oral dose comprising from 1000 to 500 000 microcapsules with modified release of AAI.

According to one variant, the medicinal formulation according to the invention comprises at least one suspension of microcapsules of AAI in an aqueous liquid phase which is preferably saturated or which becomes saturated with AAI on contact with the microcapsules, the coating of said microcapsules preferably having a composition corresponding to one of the following two families A′ and B′:
Family A′
   1A′—at least one film-forming polymer (Pol. 1′) which is insoluble in the fluids of the tract, present in a proportion of 50% to 90%, preferably 50% to 80% by weight on a dry basis relative to the total mass of the coating composition, and comprising at least one water-insoluble derivative of cellulose;
2A′—at least one nitrogenous polymer (Pol. 2′) present in a proportion of 2% to 25%, preferably 5% to 15% by weight on a dry basis relative to the total mass of the coating composition and consisting of at least one polyacrylamide and/or one poly-N-vinylamide and/or one poly-N-vinyllactam;
3A′—at least one plasticizer present in a proportion of 2% to 20%, preferably of 4% to 15% by weight on a dry basis relative to the total mass of the coating composition and consisting of at least one of the following compounds: glyceryl esters, phthalates, citrates, sebacates, cetyl alcohol esters, castor oil;
4A′—at least one surfactant and/or lubricant, present in a proportion of 2% to 20%, preferably of 4% to 15% by weight on a dry basis relative to the total mass of the coating composition and chosen from anionic surfactants and/or from nonionic surfactants and/or from lubricants; it being possible for said surfactant and/or lubricant to comprise just one or a mixture of the abovementioned products;
Family B′:
   1B′—at least one film-forming polymer which is insoluble in the fluids of the gastrointestinal tract,
   2B′—at least one water-soluble polymer,
   3B′—at least one plasticizer,
   4B′—and, optionally, at least one surfactant/lubricant, preferably selected from the group of following products:
      anionic surfactants,
      and/or nonionic surfactants.

In practice, the coating composition families A′ and B′ are, e.g., as follows:
Family A′:
   1A′—ethylcellulose and/or cellulose acetate;
   2A′—polyacrylamide and/or polyvinylpyrrolidone;
   3A′—castor oil;
   4A′—alkali metal or alkaline earth metal salt of fatty acids, stearic acid and/or oleic acid being preferred; polyoxyethylenated sorbitan esters, derivatives of polyoxyethylenated castor oil, stearates, preferably calcium stearate, magnesium stearate, aluminum stearate or zinc stearate, stearyl fumarate, preferably sodium stearyl fumarate, glycerol behenate; taken on their own or as a mixture with one another;
Family B′:
   1B′
      water-insoluble derivatives of cellulose, ethylcellulose and/or cellulose acetate being particularly preferred,
      acrylic polymers,
      polyvinyl acetates,
      and mixtures thereof;
   2B′
      water-soluble derivatives of cellulose,
      polyacrylamides,
      poly-N-vinylamides,
      poly-N-vinyllactams,
      polyvinyl alcohols (PVAs),
      polyoxyethylenes (POEs),
      polyvinylpyrrolidones (PVPs) (the latter being preferred),
      and mixtures thereof;
   3B′
      glycerol and its esters, preferably from the following subgroup: acetylated glycerides, glyceryl monostearate, glyceryl triacetate, glyceryl tributyrate,
      phthalates, preferably from the following subgroup: dibutyl phthalate, diethyl phthalate, dimethyl phthalate, dioctyl phthalate,
      citrates, preferably from the following subgroup: acetyl tributyl citrate, acetyl triethyl citrate, tributyl citrate, triethyl citrate,
      sebacates, preferably from the following subgroup: diethyl sebacate, dibutyl sebacate,
      adipates,
      azelates,
      benzoates,
      plant oils,
      fumarates, preferably diethyl fumarate,
      malates, preferably diethyl malate,
      poxalates, preferably diethyl oxalate,
      succinates, preferably dibutyl succinate,
      butyrates,
      cetyl alcohol esters,
      salicylic acid,
      triacetin,
      malonates, preferably diethyl malonate,
      castor oil (the latter being particularly preferred),
      and mixtures thereof;
   4B′
      alkali metal or alkaline earth metal salts of fatty acids, stearic acid and/or oleic acid being preferred,
      polyoxyethylenated oils, preferably polyoxyethylenated hydrogenated castor oil, polyoxyethylene-polyoxypropylene copolymers, polyoxyethylenated sorbitan esters, polyoxyethylenated castor oil derivatives,
      stearates, preferably calcium stearate, magnesium stearate, aluminum stearate or zinc stearate, stearyl fumarates, preferably sodium stearyl fumarate,
      glycerol behenate,
      and mixtures thereof.

According to an advantageous mode of this variant, in which the medicinal formulation is in suspension, it is envisioned that this suspension comprises means (b) containing “viscosifier” excipients, which are in the form of particles, each coated with at least one hydrophobic film-coating.

This hydrophobic film-coating comprises, for example, at least one product chosen from the group comprising polymers which are insoluble in the fluids of the tract.

This variant makes it possible, during a normal use, for these viscosity modifiers to remain encapsulated and therefore inactive. In the event of misuse involving crushing, the hydrophobic film-coating of these viscosity modifiers cracks, and the latter are then released and can perform their function, leading to a significant increase in viscosity, putting a stop to any misuse by injection.

Advantageously, the coating for coating (controlling the diffusion of the AAI) of the microcapsules of the suspension consists of a single layer.

This suspension contains, e.g.:
30% to 95% by weight, preferably 60% to 85% by weight of liquid phase (advantageously of aqueous solution),
5% to 70% by weight, preferably 15% to 40% by weight of microcapsules.

In practice, the amount of solvent liquid phase (preferably aqueous solution) of AAI is preferably such that the proportion of AAI dissolved and originating from the microcapsules is less than or equal to 15%, preferably less than or equal to 5% by weight relative to the total mass of AAI contained in the microcapsules.

Preferably, the liquid phase is at least partly, preferably completely, saturated with AAI subsequent to the incorporation of the microcapsules into this liquid phase.

An alternative of this suspension is for the saturation with AAI to take place by means of the AAI contained in the microcapsules.

Another alternative of this suspension is for the liquid phase to be at least partly, preferably completely, saturated with AAI by means of nonencapsulated AAI. Another alternative of this suspension is that it is in the form of a powder for oral suspension to be reconstituted: the powder contains all the elements of the suspension described above, except the water (or the liquid phase), which is added by the user.

Besides the liquid formulations, the medicinal formulation according to the invention can be in the form of a sachet of microcapsule powder, of a tablet obtained from microcapsules, or of a gelatin capsule containing microcapsules.

According to another of its aspects, the invention also encompasses the use of the microcapsules with modified release of AAI as defined above, and optionally of the microgranules with immediate release of AAI as defined above, for the preparation of pharmaceutical, microparticulate, oral galenic formulations, preferably in the form of tablets, advantageously orodispersible tablets, of powders, of gelatin capsules or of suspensions.

According to yet another of its aspects, the invention also encompasses the use of the microcapsules with modified release of AAI as defined above, and optionally of the microgranules with immediate release of AAI as defined above, for the preparation of a therapeutically safe, microparticulate oral pharmaceutical formulation designed in such a way that, once said pharmaceutical formulation has been ingested, the microcapsules that it contains are dispersed and individualized when they reach the stomach, which allows these microcapsules to be subjected to regular and gradual gastric emptying, whether the patient had eaten or was fasting at the time the dose was taken, thus guaranteeing a release of AAI within its window of bioabsorption.

The following examples illustrate the invention.

### EXAMPLES

### DESCRIPTION OF THE FIGURES ILLUSTRATING THE EXAMPLES

FIG. 1: release profiles (% by weight of AAI as a function of time in hours) for an intact tablet and for the crushed tablet and at pH 1.4.

Legend: -▪- intact tablet, -□- crushed tablet;

FIG. 2: release profiles (% by weight of AAI as a function of time in hours) for the intact and crushed microcapsules at pH 1.4.

Legend: -▴- anti-misuse microcapsules; -Δ- crushed anti-misuse microcapsules.

FIG. 3: release profiles (% by weight of AAI as a function of time in hours)of the microcapsules in 0.1 N HCl.

FIG. 4: FIG. 4A is a picture of a naked eye observation and FIG. 4B is a picture of an observation under light microscope, of the contents of a capsule according to example 8.

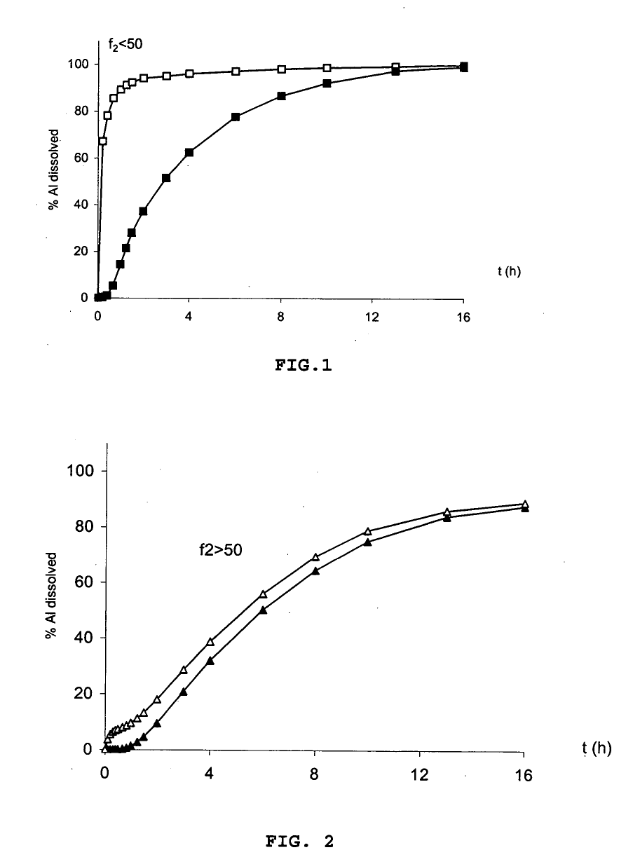

In the following examples, metformin is used as model active ingredient. Metformin hydrochloride has a solubility and a stability comparable to oxycodone hydrochloride.

### Counter-Example 1

### Tablets According to the Prior Art

Metformin tablets are prepared according to U.S. Pat. No. 5,656,295, Examples 3-4, column 10, lines 20 to 63, replacing oxycodone with metformin.

### Counter-Example 2

### Crushing of the Prior Art Tablets

A tablet of Example 1 is placed in a glass mortar and crushed. The crushed tablet is tested in a type II dissolutest in accordance with the Pharmacopoeia at 37° C. and with stirring at 75 rpm in the following media: i) solution of HCl at pH 1.4. It is noted that the release of the metformin is virtually immediate when the tablet has been crushed beforehand. The dissolution profiles are different according to the similarity factor f2 test: f2<50.

### Example 3

### Example According to the Invention

A solution of 755 g of metformin, 55.5 g of PVP and 3889 g of water is film-coated onto 216 g of neutral cellulose supports. 455 g of metformin granules are film-coated with a mixture of 147 g of ethocel 20P, 7.35 g of PVP, 7.35 g of cremophor RH 40, 34.3 g of castor oil and 2.254 kg of isopropanol. The microcapsules are then dried and sifted over 500 μm.

A mixture of 14.2 g of ethocel 20P, 1.5 g of triethyl citrate (TEC), 7.1 g of magnesium stearate, 3.51 g of PEG 6000 and 284 g of ethanol is film-coated onto 55 g of the microcapsules previously obtained. The microcapsules are then dried and sifted over 500 μm.

### Example 4

### Crushing of the Microcapsules According to the Invention

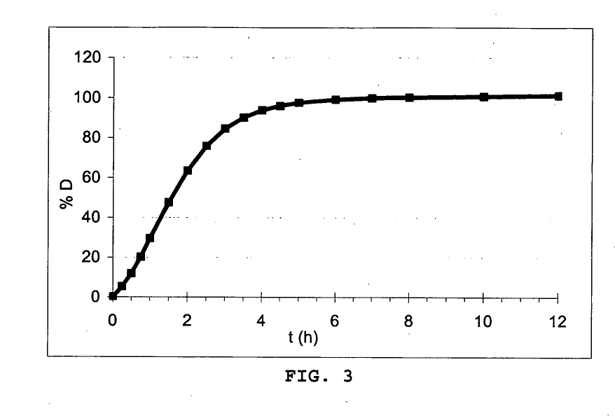

400 mg of microcapsules of Example 3 are placed in a glass mortar and crushed. The microcapsules are recovered and tested in a type II dissolutest in accordance with the Pharmacopoeia at 37° C. and with stirring at 75 rpm in a solution of HCl at pH 1.4. FIG. 2 represents the release profiles for the crushed microcapsules and for the intact microcapsules. It is noted that, in this case, the metformin release profile remains prolonged and virtually identical to the profile for uncrushed microcapsules of Example 3. The dissolution profiles are similar according to the similarity factor f2 test: f2>50.

### Example 5

### Viscosifier Mixture

The ease with which various viscosity modifiers, used alone or as a mixture, can be suctioned is reported in Table 1. The ease of suctioning was evaluated on insulin syringes having a volume of 1 ml, through a needle (29G, -0.33 mm×15 mm). The suctioning was carried out with a sterile cottonwool filter placed at the end of the needle. The medium is considered to be nonpumpable if the time required to suction 1 ml is greater than 5 min.

TABLE 1

| Compound/solvent | | Water | Vodka | 99% ethanol |
|---|---|---|---|---|
| A = Rhodigel | (40 mg/1 ml) | nonpumpable | nonpumpable | pumpable |
| | | | | (insoluble) |
| B = Ethocel | (40 mg/1 ml) | pumpable | pumpable | nonpumpable |
| 100P | | (insoluble) | (insoluble) | |
| C = Natrosol | (40 mg/1 ml) | nonpumpable | nonpumpable | pumpable |
| 250 HHX | | | | (insoluble) |
| Mixture | (3 × 40 = 120 mg/ | nonpumpable | nonpumpable | nonpumpable |
| ABC | 1 ml) | | | |

The viscosity modifiers taken separately are not soluble and viscous in all these solvents. The mixture of the viscosity modifiers makes it possible to achieve viscosities which are sufficient for the system not to be pumpable in the three media considered.

### Example 6

### Example, According to the Invention, of Particles of Viscosity Modifiers to be Incorporated into a Sachet or Suspension Formulation with a View to Preventing Misuse by Injection of a Suspension

6 g of PVP, 30 g of Rhodigel, 30 g of Ethocel 100P and 30 g of Natrosol 250 HHX are granulated with a solution of ethanol. 1 g of triethyl citrate is added, with stirring, to a solution of 8 g of Ethocel 07P, 2.1 g of stearyl alcohol and 110 g of ethanol at 70° C. After homogenization, the solution is then sprayed onto 50 g of granules obtained previously.

The Theological behavior after dispersion in water of the film-coated granules in the intact form and after crushing is reported in Table 2:

TABLE 2

| | Uncrushed film-coated | Crushed film-coated |
|---|---|---|
| | granule | granule |
| | (50 mg/1 ml) | (50 mg/1 ml) |
| Dispersion in water: | nonviscous | viscous, nonpumpable |

The combination of these particles with the microcapsules of AAI makes it possible:
1. to correctly treat patients by providing them with a suspension that is easy to swallow,
2. to combat misuse by means of a drastic increase in viscosity after crushing and suspension.

### Example 7

### Microparticles of Oxycodone HCl According to the Invention

Step 1: Granules

1615 g of oxycodone and 85 g of povidone (Plasdone® K29-32/ISP) are dispersed in a mixture containing 2052 g of water and 1105 g of ethanol. The solution is sprayed onto 300 g of cellulose spheres (Asahi-Kasei) in a Glatt GPCGl fluidized air bed.

Step 2: Anti-Crushing Microparticles

315 g of ethylcellulose (Ethocel 20 Premium/Dow), 81 g of povidone (Plasdone K29-32/ISP), 18 g of macrogol glycerol hydroxystearate (Cremophor RH40/BASF) and 36 g of castor oil (Garbit huilerie) are solubilized in a mixture composed of 3105 g of acetone and 2070 g of isopropanol. This solution is sprayed onto 450 g of granules (prepared in step 1).

The coating represents 50% of the mass of the microparticle and ensures release of the active ingredient as shown in FIG. 3. The release profile is realized under the conditions of the reference dissolution test.

### Example 8

### Content of a Gelatine Capsule According to the Invention

230 mg of microparticles obtained at the end of step 2 of Example 7, 100 mg of crushed and sieved Amberlite IR69F (sodium polystyrene sulphonate), 70 mg of sieved Polyox WSR 303 Sentry (polyethylene oxide), 3.8 mg of magnesium stearate and 1.9 mg of Aerosil 200 (colloidal silica) are introduced into a size 0 gelatine capsule.

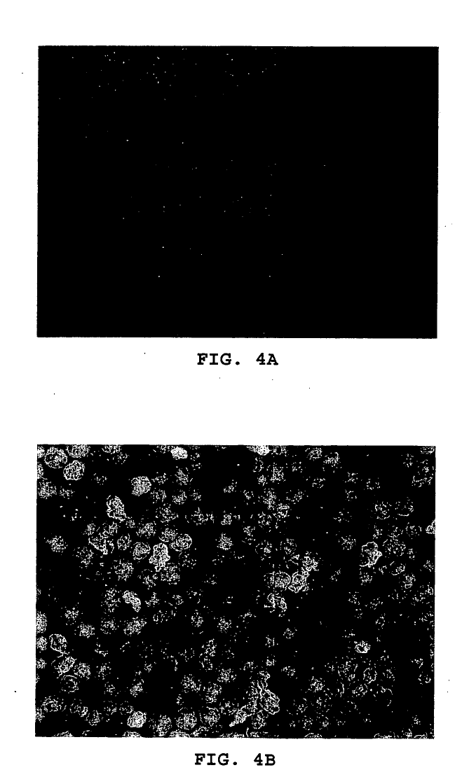

As shown in FIG. 4, with the naked eye (FIG. 4A) and under an optical microscope (FIG. 4B), the microparticles of active ingredient and the microparticles of viscosity modifier:
are indistinguishable
cannot be separated by sieving.

### Example 9

### Example of a Combination, According to the Invention

A mixture of 65 g of paracetamol, 10 g of talc, 5.5 g of PVP and 350 g of water is film-coated onto 22 g of neutral cellulose supports. A mixture of 14.2 g of Ethocel 20P, 5.1 g of PEG 6000, 1.5 g of triethyl citrate and 284 g of ethanol is film-coated onto 55 g of the microcapsules previously obtained. The microcapsules are then dried and sifted over 500 μm.

A gelatin capsule is filled with the following mixture: 300 mg of the microcapsules of paracetamol previously obtained, 15 mg of microcapsules of Example 3 and 3 mg of magnesium stearate. In the mixture thus formed, the microcapsules of paracetamol and the microcapsules of metformin cannot be discerned in terms of size, shape or color.

These microcapsules of paracetamol are immediate-release, IR, capsules. In the event of crushing in the case of an attempt at misuse, these microcapsules of paracetamol offer no resistance to the crushing, whereas the microcapsules of metformin according to the invention are protected by virtue of their overcoating (cf. Example 3 above).

1.-37. (canceled)

38. An oral medicinal formulation comprising anti-misuse means and a plurality of microcapsules with modified release of at least one analgesic active ingredient (AAI), whereby at least some of said microcapsules individually consist of a nucleus comprising at least one AAI and coated with at least one coating for modified release of the AAI; the mean diameter of said microcapsules being less than or equal to 1000 μm, preferably between 50 and 800 μm;
whereby said formulation comprises at least 1000 microcapsules per dose;
whereby the amount of AAI and the modified-release coating are such that they allow administration once or twice a day for analgesic purposes; and
whereby said anti-misuse means comprises anti-crushing means.

39. The medicinal formulation of claim 38, wherein one dose of said formulation results in a plasma profile defined as follows:
Cmax/C18h≦Cmax*/C18h*
whereby
C18h is the plasma concentration of AAI, 18h after taking the dose,
C18h* is the plasma concentration of AAI obtained under the same conditions as −C18h, with a reference immediate-release oral pharmaceutical form, containing the same dose of AAI,
Cmax is the maximum plasma concentration of AAI after taking the dose,
Cmax* is the maximum plasma concentration of AAI obtained under the same conditions as Cmax, with a reference immediate-release oral pharmaceutical form, containing the same dose of AAI.

40. The medicinal formulation of claim 39, whereby when said formulation is administered orally to an individual, the inter- and/or intraindividual standard deviation of the Cmax decreases, compared with a pharmaceutical formulation with immediate release of AAI administered to the same individual at the same dose, whereby the individual fed state or fasting state of said individual does not alter the Cmax.

41. The medicinal formulation of claim 40, wherein the factor (f) of decrease in the interindividual standard deviation of the Cmax is defined as follows: f≧1.05.

42. The medicinal formulation of claim 38, whereby when said formulation is administered orally to a sample of individuals, the mean peak/trough modulation of the plasma profiles of said AAI is less than or equal to the mean peak/trough modulation of the AAI of the same sample of individuals having received the same dose of an immediate-release of said AAI form; and a peak/trough modulation decrease factor (g) is such that: g≧1.05.

43. The medicinal formulation of claim 38, further comprising microgranules with immediate release of said AAI.

44. The medicinal formulation of claim 38, whereby 70% of said AAI is released in vitro between 1 and 24 h.

45. The medicinal formulation of claim 44, whereby said formulation has an in vitro dissolution profile such that, for any value of the time t of between 2 h and t(70%), the percentage of said AAI dissolved is greater than or equal to 35 t/t(70%).

46. The medicinal formulation of claim 38, whereby the release of said AAI is controlled by two distinct triggering mechanisms, one being based on a variation in pH and the other allowing the release of said AAI after a predetermined residence time in the stomach; whereby at constant pH 1.4 the dissolution profile contains a lag phase which lasts 7 hours or less; whereby the change from pH 1.4 to pH 7.0 results in a release phase which begins without any lag time.

47. The medicinal formulation of claim 38, further comprising at least two populations of microcapsules having different release profiles according to the similarity factor f2 test.

48. The medicinal formulation of claim 38, wherein said anti-misuse means comprises means for preventing misuse of said AAI after a possible liquid extraction.

49. The medicinal formulation of claim 38, wherein said formulation does not contain antagonist agent(s) of said AAI.

50. The medicinal formulation of claim 38, whereby said anti-crushing means comprises an overcoating for protecting the microcapsules of said AAI, whereby said overcoating has at least the following characteristics:
viscoelastic properties so as to absorb the energy dissipated during crushing,
a low cohesive strength so as to promote breaking of said overcoating and not of said microcapsules,
a low surface energy so as to promote sliding of said microcapsules during crushing,
an ability to form a paste under strong shear.

51. The medicinal formulation of claim 50, wherein said overcoating for protecting said microcapsules of said AAI is designed such that during crushing a non-immediate release from said microcapsules with modified-release of AAI is maintained.

52. The medicinal formulation of claim 50, wherein said overcoating comprises at least one film-forming compound (i) which ensures the cohesion of the overcoating, and wherein said overcoat comprises additional compounds selected from the group consisting of a lubricant/caking agent (ii), a viscoelastic compound (iii), a plasticizer (iv), and mixtures thereof.

53. The medicinal formulation of claim 52, wherein the film-forming compound (i) is selected from the group consisting of cellulose derivatives, acrylic polymers, and mixtures thereof.

54. The medicinal formulation of claim 52, further consisting of at least one lubricant/caking agent (ii) wherein said at least one lubricant/caking agent (ii) is selected from the group consisting of stearic acid and stearates, preferably calcium stearate, zinc stearate or magnesium stearate, magnesium oxide, poloxamers, sodium benzoate, anionic, cationic surfactants, nonionic surfactants, starches, corn starch, talc, colloidal silica, waxes, hydrogenated plant oils, hydrogenated cottonseed oils, hydrogenated soybean oils, hydrogenated palm oils, glycerol behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas-3, fatty bases for suppositories, glycerine, triglycerides, theobroma oils, cocoa butters, and mixtures thereof.

55. The medicinal formulation of claim 52, further consisting of at least one viscoelastic compound (iii), wherein said viscoelastic compound (iii) is selected from the group consisting of poly-N-vinylamides, gum bases, fatty alcohols, poly-N-vinyllactams, polyvinyl alcohols (PVAs), polyoxyethylenes (POEs), polyethylene glycols (PEGs), polydextroses, hydrogenated monosaccharides, hydrogenated disaccharides, hydrogenated polysaccharides, polyvinylpyrrolidones (PVPs) and mixtures thereof.

56. The medicinal formulation of claim 52, further consisting of at least one plasticizer (iv), wherein said plasticizer (iv) is selected from the group comprising: glycerol, glycerol esters, acetylated glycerides, glyceryl monostearate, glyceryl triacetate, glyceryl tributyrate, phthalates, dibutyl phthalate, diethyl phthalate, dimethyl phthalate, dioctyl phthalate, citrates, acetyl tributyl citrate, acetyl triethyl citrate, tributyl citrate, triethyl citrate, sebacates, diethyl sebacate, dibutyl sebacate, adipates, azelates, benzoates, plant oils, cottonseed oils, soybean oils, palm oils, castor oils, fumarates, diethyl fumarate, malates, diethyl malate, oxalates, diethyl oxalate, succinates, dibutyl succinate, butyrates, cetyl alcohol esters, triacetin, malonates, diethyl malonate, and mixtures thereof.

57. The medicinal formulation of claim 38, whereby said anti-crushing means comprises of: at least one excipient not contained in or supported by microcapsules, wherein said at least one excipient is capable of acting against, or even preventing, the crushing of the microcapsules of AAI, and wherein said anti-crushing means is selected from the group consisting of compression agents, inert microbeads, gum bases, viscoelastic agents, and mixtures thereof.

58. The medicinal formulation of claim 48, wherein said means for preventing misuse of said AII after a possible liquid extraction comprises at least one viscosifier excipient capable of increasing the viscosity of the extraction liquid.

59. The medicinal formulation of claim 58, wherein the location of said at least one viscosifier excipient within said formulation is selected from the group consisting of in said at least one microcapsule, on said at least one microcapsule, in said at least one overcoating of at least one microcapsule of AAI, on said at least one overcoating of at least one microcapsule of AAI, not contained in or supported by said at least one microcapsule, and mixtures thereof.

60. The medicinal formulation of claim 58, wherein said at least one viscosifier excipient is capable of increasing the viscosity of the liquid used for the possible extraction and trapping the AAI extracted in the viscous medium.

61. The medicinal formulation of claim 58, wherein said at least one viscosifier excipient is selected from the group consisting of polyacrylic acids, polyacrylic acid derivatives, polyoxyethylenes (POEs), polyvinyl alcohols (PVAs), polyvinylpyrrolidones (PVPs), gelatins, cellulose derivatives. hydroxypropylmethylcellulose, methylcellulose, hydroxyethylcellulose, carboxymethylcellulose, hydroxypropylcellulose, polysaccharides, sodium alginate, pectins, guars, xanthans, carrageenans, gellans, and mixtures thereof.

62. The medicinal formulation of claim 38, whereby said formulation cannot be converted into a dry form which can be administered by nasal inhalation.

63. The medicinal formulation of claim 38, whereby said formulation cannot be converted into an injectable form.

64. The medicinal formulation of claim 38, whereby said AAI cannot be extracted by chewing or crushing.

65. The medicinal formulation of claim 38, whereby said formulation is in the form of a single daily oral dose comprising between 1000 to 500,000 microunits containing AAI.

66. The medicinal formulation of claim 38, whereby said formulation is in the form of a single daily oral dose comprising from 1000 to 500,000 microcapsules with modified release of AAI.

67. The medicinal formulation of claim 38, whereby said formulation further comprises at least one active ingredient (AI) other than the AAI.

68. The medicinal formulation of claim 38, whereby said formulation further comprises at least one suspension of microcapsules of AAI in an aqueous liquid phase which is preferably saturated or which becomes saturated with AAI on contact with the microcapsules, whereby said coating of said microcapsules comprises
1A′ at least one film-forming polymer which is insoluble in the fluids of the tract, present in a proportion of 50% to 90% by weight on a dry basis relative to the total mass of the coating composition;
2A′ at least one nitrogenous polymer present in a proportion of 2% to 25% by weight on a dry basis relative to the total mass of the coating composition;
3A′ at least one plasticizer present in a proportion of 2% to 20% by weight on a dry basis relative to the total mass of the coating composition;
4A′ at least one surfactant or lubricant, present in a proportion of 2% to 20% by weight on a dry basis relative to the total mass of the coating composition.

69. The medicinal formulation of claim 68, wherein said
1A′ form-filming polymer comprises a water-insoluble derivative of cellulose;
2A′ said nitrogenous polymer is selected from the group consisting of polyacrylamide, poly-N-vinylamide, poly-N-vinyllactam and mixtures thereof;
3′A said plasticizer is selected from the group consisting of glyceryl esters, phthalates, citrates, sebacates, cetyl alcohol esters, castor oil and mixtures thereof;
4′A said surfactant or lubricant is selected from the group consisting of anionic surfactants, nonionic surfactants, lubricants and mixtures thereof.

70. The medicinal formulation of claim 38, whereby said formulation further comprises at least one suspension of microcapsules of AAI in an aqueous liquid phase which is preferably saturated or which becomes saturated with AAI on contact with the microcapsules, whereby said coating of said microcapsules comprises
1B′ at least one film-forming polymer which is insoluble in the fluids of the gastrointestinal tract,
2B′ at least one water-soluble polymer, and
3B′ at least one plasticizer.

71. The medicinal formulation of claim 38, further comprising at least one powder of microcapsules of AII which is reconstituted into an oral formulation by addition of water or a liquid phase.

72. The medicinal formulation of claim 58, whereby said at least one viscosifier is in the form of particles, whereby each particle is coated with at least one hydrophobic film-coating.

73. The medicinal formulation of claim 38, whereby said formulation is in the form of a sachet of microcapsule powder, a tablet obtained from microcapsules, or a gelatin capsule containing microcapsules.
